(19) United States
(12) Patent Application Publication
(10) Pub. No.: US 2026/0199150 A1
(43) Pub. Date: Jul. 16, 2026

(54) ABSORBENT ARTICLE COMPRISING A POLYMER FOAM CUSHION LAYER

(71) Applicant: The Procter & Gamble Company, Cincinnati, OH (US)

(72) Inventors: Gueltekin ERDEM, Cincinnati, OH (US); Xiaojuan SUN, Beijing (CN); Sascha KREISEL, Kriftel (DE); Abhishek SURUSHE, Mason, OH (US); Ernesto Gabriel BIANCHI, Oberursel (DE)

(21) Appl. No.: 19/450,044

(22) Filed: Jan. 15, 2026

(30) Foreign Application Priority Data
Jan. 15, 2025 (WO) PCT/CN2025/072462

(51) Int. Cl. A61F 13/535; A61F 13/15; A61F 13/53; A61F 13/539; A61L 15/24; A61L 15/42; A61L 15/60

(52) U.S. Cl. CPC A61F 13/535; A61L 15/24; A61L 15/425; A61L 15/60; A61F 2013/15373; A61F 2013/15406; A61F 2013/530591; A61F 2013/530802; A61F 2013/53908

(57) ABSTRACT

An absorbent article (20) comprising a liquid-permeable topsheet (24), a liquid-impermeable backsheet (25), an absorbent core (28) between the topsheet and the backsheet, the absorbent core comprising an absorbent material layer (30) disposed between an upper substrate layer (45) and a lower substrate layer (46), the absorbent material layer (30) comprising superabsorbent polymer particles, and a cushion layer (60) disposed between the absorbent layer (30) and the backsheet (25). The cushion layer is a polymer foam layer.

### FIELD OF THE INVENTION

The invention relates to an absorbent article for personal hygiene, such as baby diapers and adult incontinence products. The absorbent article comprises a foam-based cushion layer disposed between an absorbent layer and the backsheet.

### BACKGROUND OF THE INVENTION

Disposable absorbent articles for babies, toddlers and incontinent adults are widely used by parents and caretakers. These absorbent articles comprise an absorbent core disposed between a liquid permeable topsheet on the wearer-facing side and a liquid impermeable backsheet on the garment-facing side, among other components. The absorbent core typically comprises an absorbent layer sandwiched between an upper substrate layer and a lower substrate layer. Body fluids such as urine is acquired into the absorbent article through the topsheet and is absorbed by the absorbent layer. The absorbent layer typically comprises superabsorbent polymer (SAP) as absorbent material. The superabsorbent polymer particles may be mixed with cellulose fibers (in the context of absorbent articles often referred to as “fluff pulp” or “airfelt”). Absorbent layer free of cellulose fibers (“airfelt-free” cores) are also used in commercial products. In general, there is a trend for absorbent layer comprising ever higher proportion of superabsorbent polymer particles relative to cellulose fibers.

The backsheet typically consists of a thin laminate comprising an impermeable plastic film and outer cover nonwoven. While the backsheet film ensures impermeability of the article, the outer cover nonwoven improves the tactile sensation on the garment-facing side of the article. Superabsorbent polymer particles are relatively hard and can sometimes be felt through the backsheet by the caregivers when putting an absorbent article on a wearer. While high basis weight nonwoven materials may be used for the backsheet outer cover nonwoven to partially mask the superabsorbent particles, these materials are inherently compression sensitive and can lose some of their effectivity during the making of the article or in the packaging. Also higher basis weight cushiony material increase material costs.

There is thus a need for absorbent articles that address the above problems.

### SUMMARY OF THE INVENTION

In summary, the invention is for an absorbent article extending in a longitudinal direction, a transversal direction and a vertical direction, the article comprising a liquid-permeable topsheet, a liquid-impermeable backsheet and an absorbent core between the topsheet and the backsheet. The absorbent core comprises an absorbent material layer disposed between an upper substrate layer and a lower substrate layer. The absorbent material layer comprises superabsorbent polymer particles. According to the invention, the absorbent article comprises a polymer foam layer disposed between the absorbent layer and the backsheet that serves as a cushion layer. The cushion layer may be disposed between the lower substrate layer of the absorbent core and the backsheet.

Alternatively, the cushion layer may replace the absorbent core lower substrate layer, so that the cushion layer and the absorbent core's lower substrate layer are the same layer. In another alternative, the cushion layer may be integrated within the absorbent core, so that the cushion layer may be disposed between the absorbent layer and the lower substrate layer.

The cushion layer may consist of a single layer of polymer foam.

The cushion layer may comprise or consist of an expanded polyethylene foam layer. Alternatively, the cushion layer may be a multi-layer construction.

The absorbent article according to any of the preceding claims, wherein the cushion layer has a caliper of at least 0.5 mm, as measured at 0.85 kPa pressure according to the Caliper Measurement Method described herein.

The cushion layer may typically have a caliper in the range of 1.0 mm to 3.0 mm, as measured at 0.85 kPa pressure according to the Caliper Measurement Method described herein.

The cushion layer may typically have a basis weight in the range of from 10 g/m² to 100 g/m², preferably from 15 g/m² to 50 g/m².

Advantageous features and properties of the polymeric foam layer cushion layer and the absorbent article are further detailed in the attached claims and the following description.

### BRIEF DESCRIPTION OF THE DRAWINGS

While the specification concludes with claims particularly pointing out and distinctly claiming the present invention, it is believed that the same will be better understood from the following description read in conjunction with the accompanying drawings in which:

FIG. 1 is an exemplary absorbent article in the form of a diaper.

FIG. 2 is a transversal cross-section of the diaper of FIG. 1, wherein the cushion layer is disposed between the absorbent core's lower substrate layer and the backsheet.

FIG. 3 is an alternative transversal cross-section of the diaper of FIG. 1, wherein the cushion layer and the lower substrate layer are the same layer.

FIG. 4 is an alternative transversal cross-section of the diaper of FIG. 1, wherein the cushion layer is disposed within the absorbent core between the absorbent layer and the lower substrate layer.

### DETAILED DESCRIPTION OF THE INVENTION

### Definitions

As used herein, “absorbent article” refers to devices that absorb and contain body exudates, and, more specifically, refers to devices that are placed against or in proximity to the body of the wearer to absorb and contain any of the various exudates discharged from the body. Absorbent articles include diapers (baby and infant diapers as well as diapers for adult incontinence), pants (for babies, infants and for adults), absorbent inserts (which are intended to be inserted into an outer cover to form a diaper or pant), feminine care absorbent articles such as sanitary napkins and pantiliners, and the like. As used herein, the term “exudates” includes, but is not limited to, urine, blood, vaginal discharges, sweat and fecal matter. The absorbent articles of the present invention may be disposable absorbent articles, in particular disposable taped diapers and disposable pant diapers.

As used herein, “diaper” refers to an absorbent article generally worn by babies, infants and incontinent adults about the lower torso so as to encircle the waist and legs of the wearer and that is specifically adapted to receive and contain urinary and fecal waste. In a diaper pant, the longitudinal edges of the first and second waist region are attached to each other so that waist opening and leg openings are pre-formed. A diaper pant is placed in position on the wearer by inserting the wearer's legs into the leg openings and sliding the pant into position about the wearer's lower torso similarly to an underwear.

A pant may be pre-formed by any suitable technique including, but not limited to, joining together portions of the absorbent article using re-fastenable and/or non-refastenable bonds (e.g., seam, weld, adhesive, cohesive bond, fastener, etc.). A pant may be pre-formed anywhere along the circumference of the article (e.g., side fastened, front waist fastened).

In a taped diaper, the waist opening, and leg openings are formed when the diaper is applied onto a wearer by (releasably) attaching the longitudinal edges of the first and second waist region to each other on both sides by a suitable fastening system.

As used herein, the terms “nonwoven”, “nonwoven web” and “nonwoven layer” are used interchangeably. Nonwovens are engineered fibrous assemblies, primarily planar, which have been given a designed level of structural integrity by physical and/or chemical means, excluding weaving, knitting or paper making. The fibers may be of natural origin, such as cotton or bamboo fibers, or man-made origin. Synthetic fibers are typically made of polyolefins (such as polyethylene, polypropylene or combinations and mixtures thereof), polyethylene terephthalate (PET), co-PET, polylactic acid (PLA), polyhydroxy alkanoid (PHA), or mixtures or combinations thereof. The fibers may be staple fibers (e.g. in carded nonwovens) or continuous fibers (e.g. in spunbonded or meltblown nonwovens).

Nonwovens may be formed by many known processes such as meltblowing, spunlaying, solvent spinning, electrospinning and carding. The fibrous web can be consolidated, e.g. by hydroentanglement (in spunlaced nonwovens), air-through bonding (using hot air that is blown through the fiber layer in the thickness direction), needle-punching, one or more patterns of bonds and bond impressions created through localized compression and/or application of heat or ultrasonic energy, or a combination thereof. The fibers may also be consolidated by a binder. The binder may be binder fibers (which are subsequently molten) or may be a liquid such as a styrene butadiene latex binder. A liquid binder is provided to the fibers (e.g. by spraying, printing or foam application) and is subsequently cured to solidify.

The basis weight of nonwovens is usually expressed in grams per square meter (g/m²).

“Hydrophilic” describes surfaces of substrates which are wettable by aqueous fluids (e.g., aqueous body fluids) deposited on these substrates. Hydrophilicity and wettability are typically defined in terms of contact angle and the strike-through time of the fluids, for example through a nonwoven fabric. This is discussed in detail in the American Chemical Society publication entitled “Contact Angle, Wettability and Adhesion”, edited by Robert F. Gould (Copyright 1964). A surface of a substrate is said to be wetted by a fluid (i.e., hydrophilic) when either the contact angle between the fluid and the surface is less than 90°, or when the fluid tends to spread spontaneously across the surface of the substrate, both conditions are normally co-existing. Conversely, a substrate is considered to be “hydrophobic” if the contact angle is greater than 90° and the fluid does not spread spontaneously across the surface of the fiber.

“Longitudinal” refers to a direction running substantially perpendicular from a waist edge to an opposing waist edge of the article and generally parallel to the maximum linear dimension of the article. “Transverse” refers to a direction perpendicular to the longitudinal direction.

“Inner” and “outer” refer respectively to the relative location of an element or a surface of an element or group of elements. “Inner” implies the element or surface is oriented towards the inside of the article while “Outer” implies the element or surface is oriented towards the outside of the article.

“Body-facing” and “garment-facing” refer respectively to the relative location of an element or a surface of an element or group of elements. “Body-facing” implies the element or surface is nearer to the wearer during wear than another element of the same component. “Garment-facing” implies the element or surface is more remote from the wearer during wear than another element of the same component. The garment-facing surface may face another (i.e. other than the wearable article) garment of the wearer, other items, such as the bedding, or the atmosphere.

“Comprise,” “comprising,” and “comprises” are open ended terms, each specifies the presence of the feature that follows, e.g. a component, but does not preclude the presence of other features, e.g. elements, steps, components known in the art or disclosed herein. These terms based on the verb “comprise” encompasses the narrower terms “consisting essential of” which excludes any element, step or ingredient not mentioned which materially affect the way the feature performs its function, and the term “consisting of” which excludes any element, step, or ingredient not specified.

### General Description of an Exemplary Diaper

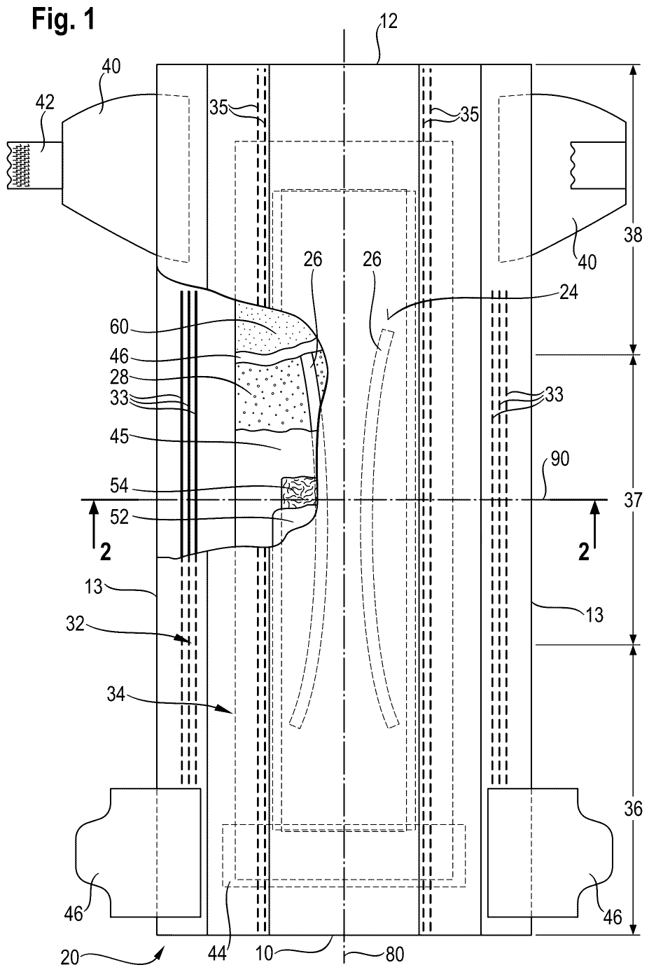

FIG. 1 is a plan view of an exemplary taped diaper 20, in a flat-out state, with portions of the diaper being cut-away to show the construction of the diaper more clearly. This diaper 20 is shown for illustration purpose only as the structure of the present invention may be comprised in a wide variety of diapers or other absorbent articles, such as pants having pre-formed side seams. The side seams of pant articles can be opened by cutting or otherwise, if it is desired to place the pant in a flattened-out configuration.

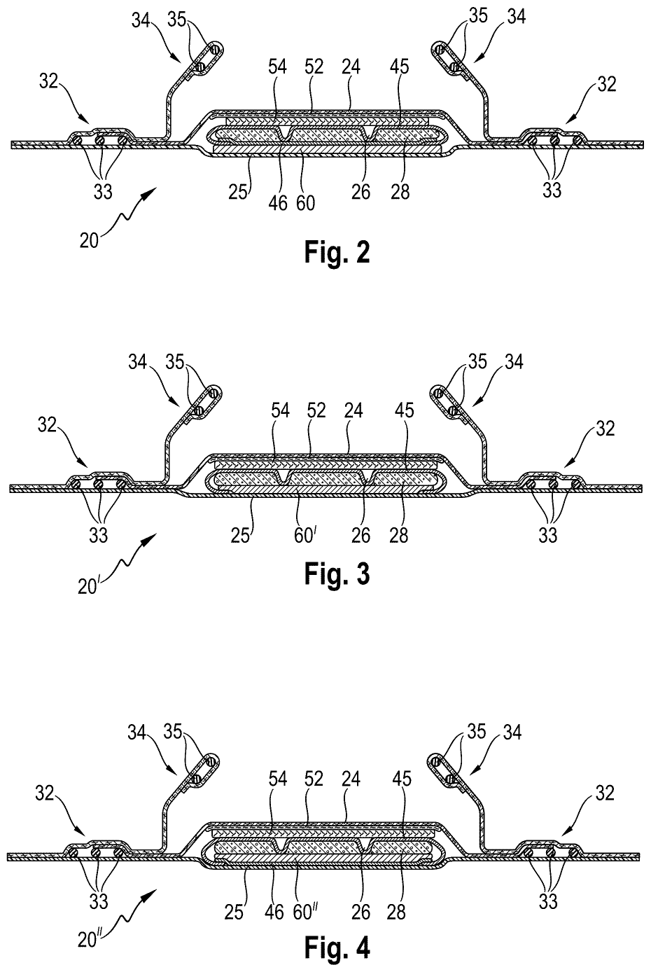

As illustrated in FIG. 1-2, the absorbent article comprises a topsheet 24, backsheet 25, and an absorbent layer 30 which is positioned between the topsheet 24 and the backsheet 25. The absorbent layer 30 has a pre-determined shape as considered in the plane formed by the transversal direction and longitudinal direction, when the article is flattened-out. The absorbent layer may be substantially rectangular as illustrated in FIG. 1. The absorbent layer may also be non-rectangular shaped, such as having tapered longitudinal edges as in a sand-hour or dog bone shape. The absorbent layer 30 may further comprise longitudinally-oriented channels 26. The core channels are typically areas substantially free of absorbent material within the absorbent layer, and that facilitate the distribution of a fluid along the length of the absorbent article.

Absorbent articles typically comprise an (upper) acquisition layer 52 directly underneath the topsheet. The absorbent article can optionally a distribution layer 54 between the acquisition layer and the absorbent core, especially when the absorbent material of the absorbent core is free of cellulose fibers. Typical acquisition layers are surfactant treated, latex bonded nonwoven acquisition layer. The distribution layer may consist of cross-linked cellulose fibers, as is known in the art. The prior art discloses many types of acquisition-distribution systems, see for example WO2000/59430, WO95/10996, U.S. Pat. No. 5,700,254, WO02/067809.

The absorbent article 20 may also comprise inner barrier leg cuffs 34 and outer leg cuffs 32, as is known in the art. The inner barrier cuffs 34 can extend upwards from the surface of the article to provide retention of the waste, while the outer cuffs are typically formed in the plane of the chassis of the article as defined by topsheet and backsheet. These cuffs are preferably elasticized, as is known in the art. The cuffs thus preferably comprise elastic threads 33, 35. Moreover, the absorbent article may comprise a fastening system, such as an adhesive fastening system or a hook and loop fastening member. The fastening system can comprise tape tabs 42 disposed on back ears 40, such as adhesive tape tabs or tape tabs comprising hook elements, cooperating with a landing zone 44 (e.g. a nonwoven web providing loops in a hook and loop fastening system). While taped diapers typically comprise back ears 40, and front ears 46, these are typically not present in pant-type absorbent articles having pre-formed side seams.

The front and/or back ears 40, 46 may be separate components attached to the absorbent article or may instead be continuous with portions of the topsheet and/or backsheet such that these portions form all or a part of the front and/or back ears 40, 46. Also combinations of the aforementioned are possible, such that the front and/or back ears 40, 46 are formed by portions of the topsheet and/or backsheet while additional materials are attached to form the overall front and/or back ears 40, 46. The front and/or back ears may be elastic or non-elastic. Also, the front ears 46 may be applied as separate components attached to the absorbent article while the back ears (or parts thereof) 40 may be continuous with portions of the backsheet and/or topsheet—or vice versa.

The longitudinal centerline 80 is the imaginary line separating the diaper along its length in two equal halves. The transversal centerline 90 is the imagery line perpendicular to the longitudinal line 80 in the plane of the flattened-out diaper and going through the middle of the length of the diaper (the same applies to for the transversal centerline and longitudinal line of other absorbent articles of the present invention). The periphery of the diaper 20 is defined by the outer edges of the diaper. The longitudinal edges 13 of the diaper may run generally parallel to the longitudinal centerline 80 of the diaper 20 and the end edges (the front waist edge 10 and the back waist edge 12) run between the longitudinal edges generally parallel to the transversal centerline 90 of the diaper 20.

The absorbent article, whether diaper or pant, can be notionally divided in front waist region 36, a back waist region 38 opposed to the first waist region 36 and a crotch region 37 located between the front waist region 36 and the back waist region 38. The front region, the crotch region and the back region each constitutes a third of the absorbent article as measured along the longitudinal centerline.

Further, the absorbent article may comprise other optional but conventional elements, which are not represented for simplicity, such as an elastic back waist feature, a front waist elastic feature, a lotion applied onto the body-facing surface of the topsheet, or a urine indicator disposed on the inner side of the backsheet that changes color when contacted with urine.

The topsheet 24, the backsheet 25 and the absorbent layer 30 may be assembled in a variety of well-known configurations, in particular by gluing, heat embossing, ultrasonic bonding or combinations thereof. Exemplary diaper configurations are described generally in U.S. Pat. Nos. 3,860,003; 5,221,274; 5,554,145; 5,569,234; 5,580,411; and 6,004,306.

The topsheet 24 is a liquid permeable layer that is in contact with the wearer's skin permitting liquid bodily exudates to readily penetrate into the absorbent article. Typical topsheet material are nonwovens and apertured plastic films, but other materials are possible such as porous foams, reticulated foams, woven materials etc. Suitable nonwovens may comprise natural fibers (e.g., wood or cotton fibers), synthetic fibers (e.g., polypropylene or bicomponent PE/PP fibers or mixtures thereof), or a combination of natural and synthetic fibers. The topsheet may consist of a single layer, or may be a laminate or a composite of different materials. The topsheet may be apertured or non-apertured. The topsheet may also have three-dimensional features, and/or may have a plurality of embossments (e.g., a bond pattern). Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet.

The backsheet 25 is generally that portion of the absorbent article 20 that constitutes all or a part of the garment-facing surface of the absorbent article. The backsheet 25 may be joined at least partially to the topsheet 24, the absorbent layer 30, the substrate layer 46, or the cushion layer 60, by any attachment methods known to those of skill in the art in particular adhesive and/or fusion and/or ultrasonic bonding. The backsheet 25 prevents, or at least inhibits, the bodily exudates absorbed and contained in the absorbent layer 30 from soiling articles such as bedsheets, undergarments, and/or clothing. The backsheet is typically liquid impermeable, or at least substantially liquid impermeable.

The backsheet 25 is typically comprised of a thin impermeable plastic film, usually a thermoplastic film having a thickness of about 0.01 mm to about 0.05 mm. The backsheet film material may be breathable, which permit vapors to escape from the absorbent article, while still preventing, or at least inhibiting, bodily exudates from passing through the backsheet. A breathable backsheet may have a Water Vapor Transmission Rate (WVTR) of from 1,000 to 15,000 g/m²/24 h, or from 1,000 to 10,000 g/m²/24 h, or from 1,500 to 10,000 g/m²/24 h as measured using a PERMATRAN-W Model 101K (available from Mocon, Inc., Minneapolis, MN) or equivalent, according to Nonwovens Standard Procedure NWSP 70.4.R0(15) with the following specifications: experiments were carried out in a lab controlled at 23° C.±2 C.° and 50% RH±2% RH and the instrument cells heated to 37.8° C. (100° F.).

The backsheet 25 may also comprise a backsheet outer cover nonwoven (not represented separately) to improve the touch of the backsheet. The backsheet outer cover nonwoven is typically a thin nonwoven material that is adhesively joined to the outer surface of the backsheet film. The backsheet outer cover nonwoven forms the outermost garment-facing surface of the backsheet. The backsheet outer cover nonwoven may comprise a bond pattern, apertures, and/or three-dimensional features.

The absorbent layer 30 typically comprises superabsorbent polymer particles. The superabsorbent particles may be mixed with cellulose fibers. Alternatively, the absorbent layer may be free of cellulose fibers mixed with the superabsorbent polymer particles. Such absorbent cores where the superabsorbent polymer particles are not mixed with cellulose fibers are designated in the art as airfelt-free cores. In such airfelt-free cores, the superabsorbent polymers particles may be immobilized by a glue on a core substrate, in particular a microfibrous glue, or alternatively supported within a high loft, porous nonwoven, as is known in the art.

The absorbent layer 30 is typically disposed between an upper substrate layer 45 oriented towards the topsheet and a lower substrate layer 46 oriented towards the backsheet. The absorbent layer and the substrate layers together forming the absorbent core. The upper substrate and lower substrate layer are commonly referred to respectively as core cover and dusting layer, and together as core wrap. These substrate layers are typically low basis weight nonwoven, (typically in the range of from 10 gsm to 20 gsm, in particular less than 14 gsm). Core wrap layers are typically SMS nonwovens (Spunbond-Meltblown-Spunbond laminate) are known in the art. The upper and lower substrate layer 45, 46 may be any material capable of providing a support for the absorbent layer.

The substrate layers may be made of the same or different materials, i.e. two nonwoven webs which have the same of different properties. The upper and the lower substrate layers 45, 46 may be made of two discrete layers, as represented in the Figures. The substrate layers are preferably bonded longitudinally to prevent the absorbent layer from being released sideways. The substrate layers may also be optionally bonded transversally at the front and the back of the absorbent core. The substrate layers may be bonded in a C-wrap configuration where one of the top or bottom substrate layer is larger than the other, so that flaps can be folded around the absorbent layer and attached to the other substrate, but other bonding configurations are possible, in particular a face to face bonding configuration. Alternatively, the core wrap may be made of a single material, i.e. of the same nonwoven, which is wrapped around the absorbent layer.

The upper substrate layer 45 and the lower substrate layer 46 typically least partially or fully enclose the absorbent layer 30, providing for dry and wet immobilization of the absorbent layer. Additionally, the absorbent layer may be immobilized on the upper substrate layer 45 and/or on the lower substrate layer 46, and/or on cushion layer 60, for example by use of hot melt adhesive.

The lower substrate layer 46 is typically provided between the absorbent layer 30 and the lower cushion layer 60 as represented in FIG. 2. In other words, the cushion layer is typically disposed between the lower substrate layer 46 and the liquid-impermeable backsheet 25. This construction is also the simplest to make, as it does not require making changes to existing absorbent core making process, the cushion layer being additionally interposed between the absorbent core and the backsheet.

Alternatively, the cushion layer 60′ may also serve as the lower substrate layer 46 and be in direct contact with the absorbent layer 30 (so that there is no separate lower substrate layer 46). In this case, the upper substrate layer 45 and the cushion layer 60 may partly or fully enclose the absorbent layer 30. This embodiment is represented in FIG. 3 for alternative absorbent article 20′. This construction may require the cushion layer 60′ to be larger and longer than the configuration of FIG. 2 to cover the entire absorbent layer 30, which may have additional material costs if the cushion layer is more expensive than a typical SMS nonwoven for example.

In another alternative, as illustrated in FIG. 3, the absorbent layer 30 and the cushion layer 60″ may be partially or fully enclosed within the absorbent core between the upper substrate layer 45 and lower substrate layer 46. The cushion layer 60″ is disposed in this alternative between the absorbent layer 30 and the lower acquisition substrate layer 46.

The absorbent layer 30 comprises superabsorbent polymer particles. The absorbent layer may optionally comprise cellulose fibers, or alternatively be free of such cellulose fibers. The absorbent layer may comprise, by weight of the absorbent layer, at least 30%, or at least 40%, or at least 50%, or at least 60%, or at least 70%, or at least 80%, or at least 90% of superabsorbent polymer particles. The absorbent layer may comprise, by weight of the absorbent layer, less than 50% of cellulose fibers, in particular less than 25%, or less than 20%, or less than 15% or less than 10 weight % of cellulose, or less than 5% by weight of cellulose fibers, and may be free of cellulose fibers.

Various absorbent core designs comprising high amounts of SAP have been proposed in the past, see for example in U.S. Pat. No. 5,599,335 (Goldman), EP1,447,066 (Busam), WO95/11652 (Tanzer), US2008/0312622A1 (Hundorf), WO2012/052172 (Van Malderen). In particular, the SAP printing technology as disclosed in U.S. Pat. No. 7,838,722 (Blessing), U.S. Pat. Nos. 9,072,634 and 8,206,533 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate, such as a carded nonwoven layer, having a porous structure into which SAP particles have been deposited, may also be used in present disclosure.

When the absorbent layer is free of cellulose fibers, the absorbent layer may consist of the superabsorbent polymer particles. The resulting absorbent layer has a reduced thickness in the dry state compared to conventional absorbent cores including cellulosic fibers. The reduced thickness helps to improve the fit and comfort of the absorbent article for the wearer.

The absorbent layer 30 defines a deposition area, when considered in the plane of the absorbent article as shown in FIG. 1, having a pre-determined shape. The absorbent layer 30 may have any shape, in particular a rectangular shape as illustrated in FIG. 1, but other shapes are common such as dog-bone or sand-hour shaped having a tapering in the crotch region of the article.

The absorbent layer 30 may define one or more channel(s) 26, where substantially no absorbent material is present, apart from possible accidental discrete contamination. These absorbent material-free channels preferably do not extend to any of the side of the absorbent material layer. The channels may thus be completely surrounded by absorbent material. The channels are typically elongated in the longitudinal direction, having a length of from 20% and 80%, or from 20% to 70%, or from 30% to 60%, by total longitudinal dimension of the absorbent material layer 30. The channel may be straight, curved, or combinations thereof. The one or more channels are typically symmetrically disposed relative to the longitudinal axis When a pair of channels is present, these may be discrete, i.e. not connected with another, as illustrated in FIG. 1, or alternatively the channels 26 may be connected at one or both their extremities to form a U or O shape. Examples of such absorbent material-free channels are disclosed in further details in WO2012170778A1, WO2012170781 (Kreuzer et al.).

The upper substrate layer 45 and lower substrate layer 46 may be bonded to each other through at least a portion of the length of the channel. This bond provides for structural integrity of the channels in dry and wet state. Any known bonding techniques known in the art may be used to provide for this bond. The channel bonding of the core wrap layers may be selected from adhesive bonding, thermo bonding, mechanical bonding, ultrasonic bonding, or any combinations thereof. An adhesive may be for example applied in the areas of the channels on the inner side of the top side and/or the inner side of the bottom side of the core wrap, typically by slot glue application or any other means, followed by the application of pressure in the areas of the channels to provide a good adhesive bonding in these areas. Exemplary patent disclosures of such adhesive bonding processes can be found for an airfelt or airfelt-free absorbent cores in WO2012/170798AI (Jackels et al.), EP2,905,000 (Jackels et al.), WO2016/149251A1 (Stiehl et al.) and EP2,905,001 (Armstrong-Ostle et al.).

Other bonding such as thermo bonding, mechanical bonding, ultrasonic bonding can also be used as additional bonding or as an alternative bonding. For example, an adhesive bonding may be reinforced by a thermo bonding, mechanical bonding or ultrasonic bonding. Such thermo, mechanical or ultrasonic bonding can be applied on the channels through the external sides of the core wrap substrates.

Typically, the bonds may generally have the same outline and shape as the channels 26 in which they are contained but may be slightly smaller to allow for a safety margin (e.g. by a few mm) as some deviations from the optimal registration may happen during high-speed process. The absorbent material free channels may also be not bonded or have one or more section which is bonded and one or more section that is not bonded.

Suitable SAP may be any water-insoluble, water-swellable polymers capable of absorbing large quantities of fluids, as is known in the art. “Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test as indicated in EDANA method NWSP 241.0.R2 (19). The SAP may in particular have a CRC value of at least 20 g/g, in particular of from about 20 g/g to about 40 g/g. “Superabsorbent polymer particles”, as used herein, refers to a superabsorbent polymer material which is in particulate form such as irregular shape granules, spherical particles, flakes, so as to be flowable in the dry state.

### Cushion Layer 60

The cushion layer 60 is disposed between the absorbent layer 30 and the backsheet 25. If the absorbent article comprises a lower substrate layer 46 that, in conjunction with an upper substrate layer 45 at least partially encloses the absorbent layer 30, the cushion layer may be typically disposed in direct contact between the backsheet 24 and the lower substrate layer 45 as illustrated in FIG. 2. However other configurations are possible, as illustrated in FIGS. 3 and 4 as already discussed above, for example.

According to the present invention, the inventors have found that a polymer foam can serve as a cushion layer to reduce or eliminate the grainy feel felt through the backsheet caused by superabsorbent particles in the absorbent material layer.

As is known in the art, polymer foam is a lightweight, cellular material formed by the expansion of a polymer matrix, which creates a network of interconnected gas-filled cells. This foam structure can be achieved through various manufacturing processes, such as extrusion, molding, or batch foaming, often involving the use of physical or chemical blowing agents. Commonly used polymers include thermoplastics like polyethylene, polypropylene, and polystyrene, as well as thermosetting polymers like polyurethane. The unique architecture of polymer foams provides a combination of desirable properties, including low density, high strength-to-weight ratio, and excellent thermal and acoustic insulation, making them suitable for a wide range of applications.

Preferred polymers for the cushion layers are polyolefin-base foams, in particular closed-cell foam material. Expanded Polyethylene (EPE) foam is an exemplary closed-cell foam material which may be preferred due to its availability, stability and safety.

The cushion layer may be comprised of a single foam layer. Alternatively, it is not excluded that the cushion layer may be a multi-layer construction such as a laminate or an integrated layer comprising integrated sub-layers, as long as the multi-layer construction has at least one polymeric foam layer.

A particular example of EPE foam is described and characterized in the experimental section below. However other foam layers may be used in the invention. Advantageous properties of the cushion layer of the invention are indicated below.

Additional layers provided to an absorbent article generally increase the thickness and bulk of the article, thereby reducing wearer comfort. Also, increased bulk is generally not desirable, especially between the wearer's legs. Therefore, it may be desirable to limit the caliper of the cushion layer to be in the range of from 0.5 mm up to 4 mm, in particular in the range of from 1 mm to 2 mm, as measured at 0.85 kPa pressure according to the Caliper Measurement Method described herein.

The basis weight of the cushion layer may typically relatively low, and in the range of from 10 g/m² to 60 g/m², or from 15 g/m² to 40 g/m², for example 20 g/m². The basis weight of the cushion layer, at least when comprised of a single layer, is typically homogeneous throughout the length and width of the cushion layer (i.e. in the longitudinal and transverse direction). The basis weight of a material is typically provided by the supplier, and if not can be calculated by dividing the weight of the cushion layer by its surface.

The density of the cushion layer may typically be in the range of from 0.5 g/cm³ to 3 g/cm³ as measured at 0.85 kPa pressure according to the Caliper Measurement Method described herein.

The cushion layer may have any dimensions, balancing material usage with the cushiony effect desired. As a good balance, the cushion layer may be as long as the absorbent core in the longitudinal direction but shorter in the lateral direction but other configurations are possible. The cushion layer may be typically free of superabsorbent polymer.

### Packages

A plurality of articles according to the invention may be comprised in a package for transport and sale. At least 50% of the articles, and preferably all the articles, in the package may be according to the invention. The absorbent articles may be folded and packaged as is known in the art. The package may be for example a plastic bag or a cardboard box. Diapers may typically bi-folded along the transversal axis and the ears folded inwardly before being packaged. The absorbent articles may be packed under compression so as to reduce the size of the packages, while still providing an adequate number of absorbent articles per package. By packaging the absorbent articles under compression, caregivers can easily handle and store the packages, while also providing distribution and inventory savings to manufacturers owing to the size of the packages.

The absorbent articles may thus be packaged compressed at an In-Bag Compression Rate of at least 10%, in particular of from 10% to 50%, in particular from 20% to 40%. The “In-Bag Compression Rate” as used herein is one minus the height of a stack of 10 folded articles measured while under compression within a bag (“In-Bag Stack Height”) divided by the height of a stack of 10 folded articles of the same type before compression, multiplied by 100; i.e. (1-In-Bag Stack Height/stack height before compression)*100, reported as a percentage. Of course, the stack in the bag does not need to have exactly 10 articles, rather the value measured for the height of stack of article in the package is divided by the number of articles in the stack and then multiplied by 10. The method used to measure the In-Bag Stack Height is described in further details in the Test Procedures. The articles before compression are sampled from the production line between the folding unit and the stack packing unit. The stack height before compression is measured by taking 10 articles before compression and packing and measuring their stack height as indicated for the IBSH.

Absorbent article packages of the present disclosure may in particular have an In-Bag Stack Height of less than 110 mm, less than 105 mm, less than 100 mm, less than 95 mm, less than 90 mm, specifically reciting all 0.1 mm increments within the specified ranges and all ranges formed therein or thereby, according to the In-Bag Stack Height Test described herein. For each of the values indicated in the previous sentence, it may be desirable to have an In-Bag Stack Height of greater than 60, or greater than 70 mm, or greater than 75 mm, or greater than 80 mm. Alternatively, packages of the absorbent articles of the present disclosure may have an In-Bag Stack Height of from 60 mm to 110 mm, from 65 mm to 110 mm, from 70 mm to 110 mm, from 75 mm to 105 mm, or from 80 mm to 100 mm, specifically reciting all 0.1 mm increments within the specified ranges and all ranges formed therein or thereby, according to the In-Back Stack Height Test described herein.

### Bio-Sourced Materials

Components of the disposable absorbent article (i.e., diaper, pant, sanitary napkin, pantiliner, etc.) of the present invention can at least partially be comprised of bio-sourced content as described in US 2007/0219521A1 Hird et al published on Sep. 20, 2007, US 2011/0139658A1 Hird et al published on Jun. 16, 2011, US 2011/0139657A1 Hird et al published on Jun. 16, 2011, US 2011/0153012A1 Hird et al published on Jun. 23, 2011, US 2011/0139662A1 Hird et al published on Jun. 16, 2011, and US 2011/0139659A1 Hird et al published on Jun. 16, 2011. These components include, but are not limited to, topsheet nonwovens, backsheet films, backsheet nonwovens, barrier leg cuff nonwovens, superabsorbent layer, upper and lower substrate layer, adhesives, fastener hooks, and fastener landing zone nonwovens and film based.

The disposable absorbent article component may comprise a bio-based content value from about 10% to about 100% using ASTM D6866-10, method B, in another embodiment, from about 25% to about 75%, and in yet another embodiment, from about 50% to about 60% using ASTM D6866-10, method B.

In order to apply the methodology of ASTM D6866-10 to determine the bio-based content of any disposable absorbent article component, a representative sample of the disposable absorbent article component must be obtained for testing. Thereto, the disposable absorbent article component may be ground into particulates less than about 20 mesh using known grinding methods (e.g., Wiley® mill), and a representative sample of suitable mass taken from the randomly mixed particles.

### Examples

### Raw Material Data

The following are examples and counter-examples for a cushion layer.

Invention Example 1 is an E-PE foam layer having a basis weight of 20 g/m² (ex. YanJan, Xia'men, China).

Comparative examples 1-3 are conventional nonwovens having a higher basis weight (40 g/m² or 60 g/m²).

The calipers at different pressure, % recovery and Handle-o-meter value, which quantifies the flexibility and cushiness of sheeted materials including nonwovens, tissue, toweling, film, and textiles, were measured and are reported in Table 1 below.

TABLE 1

| | | Material Type | | | |
|---|---|---|---|---|---|
| | | | | Carded | Carded |
| | | | | Calander | Calander bond - |
| | | E-PE | Carded ATB | bond | semi philic |
| | | Code | | | |
| | | Inv. Ex. 1 | Comp. Ex. 1 | Comp. Ex. 2 | Comp. Ex. 3 |
| Basis weight [gsm] | | 20 | 60 | 40 | 40 |
| 1) Caliper (40 mm footer/ | Avg (stdv) | 1.37 (0.02) | 1.12 (0.21) | 0.76 (0.03) | 0.74 (0.06) |
| 0.85 kPa) [mm] | | | | | |
| 2) Caliper (40 mm footer/ | Avg (stdv) | 1.22 (0.01) | 0.53 (0.02) | 0.44 (0.02) | 0.42 (0.03) |
| 5.0 kPa) [mm] | | | | | |
| 3) Caliper (40 mm footer/ | Avg (stdv) | 1.06 (0.01) | 0.40 (0.02) | 0.33 (0.01) | 0.32 (0.02) |
| 15.4 kPa) [mm] | | | | | |
| 4) Caliper, measured | Avg (stdv) | 1.31 (0.02) | 0.72 (0.07) | 0.62 (0.03) | 0.62 (0.05) |
| after 5 seconds of | | | | | |
| caliper compression | | | | | |
| with 15.4 kPa (40 mm | | | | | |
| footer/0.85 kPa) [mm] | | | | | |
| % Caliper/Recovery | Avg (stdv) | 0.95 (0.01) | 0.66 (0.15) | 0.81 (0.03) | 0.84 (0.01) |
| between Caliper 1) and | | | | | |
| Caliper 4) after | | | | | |
| compression with 15.4 | | | | | |
| kPa [%] | | | | | |
| Mean Handle-o-meter | Avg (stdv) | 100.0* (0.0) | 30.5 (3.1) | 15.7 (1.0) | 15.0 (1.5) |
| (100 × 100) CD1 [g] | | | | | |
| Mean Handle-o-meter | Avg (stdv) | 85.7 (3.5) | 43.9 (4.3) | 9.9 (0.8) | 10.2 (1.0) |
| (100 × 100) MD1 [g] | | | | | |

*100 g is load cell limit/all measurements >100

As can be seen from Table 1, the E-PE foam had a higher caliper under pressure, better recovery and better flexibility than the comparative conventional nonwovens, despite having a lower basis weight.

### Examples of Absorbent Article

The material above were used as cushion layer in a taped diaper of similar construction as represented in FIGS. 1-2. For this purpose, Commercial Size 4 Pampers® Baby Dry market product (Western Europe, 2H 2020) were modified to include a cushion layer according to the example materials above. In addition, the non-modified Size 4 Pampers Baby Dry diaper was also used as comparative example.

Description of the modified absorbent article: the topsheet, dual acquisition and distribution layers and the absorbent core of the Pampers diapers were carefully removed using ice-spray. For each diaper, the cushion layer was attached to the wearer-facing surface of the backsheet with a hotmelt adhesive applied in form of spirals with a basis weight of 5 g/m². The cushion layer was cut with a width of 90 mm and was centered on the backsheet with respect to the transverse direction. The cushion layer had the same length as the original absorbent core and was placed at the same position as the original absorbent core with respect to the longitudinal direction. The original absorbent core was then attached to the cushion layer with a double sided tape with a basis weight around 22.5 g/m² at the center (12 mm wide, continuous length) and close to each corners of the cushion layer in longitudinal direction (40 mm long and 12 mm wide) such that the absorbent core and the dual acquisition layer were placed in the exact position as before removal. The absorbent core was attached to the cushion layer in the longitudinal direction at the sides of the cushion layer, with the front and back edges of the cushion layer corresponding to the front and back edges of the absorbent core. Finally, the combination of original topsheet and dual acquisition-distribution layers were re-attached to the absorbent core with a hot melt adhesive applied in form of spirals with a basis weight of 5 g/m². The diaper samples were compacted in a flexible bag at an In Bag Stack Height, i.e. the total caliper of 10 bi-folded diapers, of 78 mm for 1 week. Then the bag was opened and the diapers out of the bag were conditioned at least 24 hours prior to any testing at 23° C.+/−2° C. and 50%+/−10% Relative Humidity (RH).

The diapers were then tested in an expert panel for grainy feel and cushiony feel as reported in Table 2 below.

TABLE 2

| | | | Finished Product data | |
|---|---|---|---|---|
| | | | Sensory Attributes | |
| | | | Grainy Feel | Cushiony Feel |
| Option | Cushion Layer Material Description | | (Lower is better) | (Higher is better) |
| A | Inv. Ex. 1 (E-PE) | 20 gsm BW/1 mm Caliper | 27.3 | 16.5 |
| B | Comp. Ex. 1: | 60 gsm | 38.9 | 15.1 |
| | Carded | | | |
| | Airthrough | | | |
| | bonded | | | |
| C | Comp. Ex. 2: | 40 gsm Round PET/trilobal | 41.8 | 15.8 |
| | Carded, | PP fiber- pseudo philic/both | | |
| | calendered | fibers surfactant treated | | |
| | bonded | | | |
| D | Comp. Ex. 3: | 40 gsm Round PET/trilobal | 43.6 | 16.8 |
| | Carded, | PP fiber- semi philic (only PP | | |
| | calendered | fibers treatment with | | |
| | bonded | surfactant) | | |
| E | Current market | No cushion layer | 51.0 | 12 |

As can be seen in Table 2 above, the diapers with the cushion layer of the invention had better sensory attributes than the comparative diapers.

### Test Methods

### Caliper Measurement Method

The caliper of the cushion layer is determined using the Caliper Test Method. In the Caliper Test Method, two flat, parallel surfaces are used to apply unidirectional pressure to both sides of a substrate specimen, and the resulting separation between the parallel surfaces is measured. All measurements are performed in a laboratory maintained at 23±2° C. and 50±2% relative humidity and test specimens are conditioned in this environment for at least 2 hours prior to testing.

One suitable example of apparatus for use in the Caliper Method is a Mitutoyo Digimatic Series 543 ID-C digital indicator (Mitutoyo America Corp., Aurora, Illinois, USA), or equivalent, fitted with a circular flat “foot” having a diameter of 4.0 cm at the end of the moving shaft of the indicator gauge. The indicator is mounted on a horizontal granite base such that the shaft of the indicator gauge is oriented vertically, and the plane of the circular foot is parallel to the granite base. The circular foot is sized and weighted such that the gravitational force associated with the mass of the foot and the indicator shaft together divided by the area of the circular foot constitutes 0.85 kPa of downward pressure from the circular foot on the granite base (other weight can be used to measure the caliper at different pressure as desired, e.g. 5.0 kPa, 15.4 kPa as indicated in Table 1). Specimens at least as large as the circular foot are analyzed between the circular foot and granite base.

The caliper is measured 2 s after the round foot has been contacted with the specimen. For a given material, 10 specimens from this material are measured and the average value reported as the caliper of the material.

### Handle-o-Meter Test Method

The Handle-o-meter Test Method measures the force required to push a specimen into a slot of predetermined width with a metal blade working at a predetermined capacity.

The Handle-o-meter test method is carried out as indicated in ASTM D6828-02 (reapproved 2019). At least 5 samples are measured and the values averaged.

The Mean Handle-o-meter value of the fabric is expressed in g, based on the load max (g) determined with the Handle-o-meter (sum of 4 load max measurements as indicated in ASTM D6828-02).

### Misc.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An absorbent article (20, 20′, 20″) extending in a longitudinal direction (80), a transversal direction (90) and a vertical direction, the article comprising:
a liquid-permeable topsheet (24);
a liquid-impermeable backsheet (25);
an absorbent core (28) between the topsheet and the backsheet, the absorbent core comprising an absorbent material layer (30) disposed between an upper substrate layer (45) and a lower substrate layer (46), the absorbent material layer comprising superabsorbent polymer particles; and
a cushion layer (60, 60′, 60″) disposed between the absorbent layer (30) and the backsheet (25);
wherein the cushion layer is a polymer foam layer.

2. The absorbent article according to claim 1, wherein the polymer foam layer is a closed-cell polymer foam layer.

3. The absorbent article according to claim 1, wherein
the cushion layer (60) is disposed between the lower substrate layer (46) and the backsheet (25); or
the cushion layer (60′) and the lower substrate layer (60′) are the same layer; or
the cushion layer (60″) is disposed between the absorbent layer (30) and the lower substrate layer (46).

4. The absorbent article according to claim 1, wherein the cushion layer (60, 60′, 60″) has a caliper of at least 0.5 mm, as measured at 0.85 kPa of pressure according to the Caliper Measurement Method described herein.

5. The absorbent article according to claim 1, wherein the cushion layer (60, 60′, 60″) has a caliper in the range of 1.0 mm to 3.0 mm, as measured at 0.85 kPa of pressure according to the Caliper Measurement Method described herein.

6. The absorbent article according to claim 1, wherein the cushion layer has a basis weight in the range of from 10 g/m² to 100 g/m².

7. The absorbent article according to claim 1, wherein the cushion layer (60, 60′, 60″) consists of a single foam layer.

8. The absorbent article according to claim 1, wherein the cushion layer (60, 60′, 60″) comprises or consists of a layer of expended polyethylene (E-PE) foam.

9. The absorbent article according to claim 1, wherein the density of the cushion layer is in the range of from 0.5 g/cm³ to 3 g/cm³ as measured at 0.85 kPa of pressure according to the Caliper Measurement Method described herein.

10. The absorbent article according to claim 1, wherein the length of the cushion layer is at least as long as the length of the absorbent core, as measured in the longitudinal direction.

11. The absorbent article according to claim 1, wherein the absorbent layer does not comprise cellulose fibers mixed with superabsorbent polymer particles.

12. The absorbent article according to claim 1, wherein the absorbent layer (30) comprises or consist of superabsorbent polymer particles that are immobilized on at least one or both of the upper substrate layer (45) or the lower substrate layer (46) by an adhesive, in particular a microfibrous adhesive.

13. The absorbent article according to claim 1, wherein the absorbent layer comprises at least one channel (26) which is free of superabsorbent particles.

14. The absorbent article according to claim 13, wherein the at least one channel is at least partially longitudinally-extending.

15. The absorbent article according to claim 13, wherein the upper substrate layer (45) and the lower substrate layer (46) are bonded through the channel.

16. The absorbent article according to claim 1, the article further comprising an acquisition layer (52) between the topsheet (24) and the upper substrate layer (45).

17. The absorbent article according to claim 16, the article further comprising a distribution layer (54) between the acquisition layer (52) and the absorbent core (28).

18. The absorbent article according to claim 1, wherein the super absorbent polymer particles are made of cross-linked polymeric materials and can absorb at least 10 times their weight of an aqueous 0.9% saline solution as measured using the EDANA method WSP 241.2.R3 (12).
